(19) United States
(12) Patent Application Publication
(10) Pub. No.: US 2021/0040500 A1
(43) Pub. Date: Feb. 11, 2021

(54) ENGINEERED MICROVESICLES AND EXOSOMES FOR IMMUNOMODULATION

(71) Applicant: UNIVERSITY OF WYOMING, Laramie, WY (US)

(72) Inventors: Jared BUSHMAN, Laramie, WY (US); Kelly Cristine SANTOS ROBALLO, Laramie, WY (US); Jason GIGLEY, Laramie, WY (US)

(21) Appl. No.: 16/988,878

(22) Filed: Aug. 10, 2020

Related U.S. Application Data
(60) Provisional application No. 62/884,618, filed on Aug. 8, 2019.

(51) Int. Cl. C12N 15/86; C12N 15/90

(52) U.S. Cl. CPC C12N 15/86; C12N 2310/20; C12N 15/907

(57) ABSTRACT

The disclosure generally relates to a method for fabrication of extracellular vesicles derived from antigen-presenting cells engineered to create allospecific tolerance for tissue transplantation. The display of both donor MHCs and co-inhibitory receptors on the same EV creates a synergistic effect on host T cells, preventing activation of naïve T cells against a foreign graft and inducing anergy of previously activated anti-graft T cells. Embodiments of the disclosure demonstrate an allospecific immunomodulatory strategy to avert the need for chronic, toxic immunosuppression in reconstructive transplantation and VCA. One embodiment includes a novel cell and virus-free system to display diverse alloantigens with tolerance-inducing stimuli.

### CROSS-REFERENCE TO RELATED APPLICATIONS

This application claims priority to U.S. Provisional Patent Application Ser. No. 62/884,618 filed on Aug. 8, 2019, which is incorporated herein by reference in its entirety.

### BACKGROUND

### Field

Embodiments of the present disclosure generally relate to methods for fabricating extracellular vesicles. More specifically, embodiments of the disclosure relate to fabrication of extracellular vesicles derived from antigen-presenting cells (APCs) that are engineered to dampen the immune response and prevent T cell activation against a foreign graft.

### Description of the Related Art

Reconstructive transplantation and vascularized composite allotransplantation (VCA) restores quality of life for individuals who have suffered devastating injuries. In 2017, twenty-eight VCAs were performed in the United States, with tissue procured from twenty-one donors. These numbers are small, in comparison with the hundreds of thousands of patients who lose limbs due to trauma and disease each year in the United States, for at least the reason that VCA carries substantial risk. VCA is accompanied by an aggressive lifelong, immunosuppressive therapy that is toxic, expensive, and increases a patient's susceptibility to pathogens. Such immunosuppressive therapy includes, e.g., immunosuppressive drugs, that patients typically take to prevent rejection over the course of their lifetime.

Unfortunately during allogeneic transplantation, a recipient's immune system remains one of the most arduous barriers to graft transplantation and acceptance as a routine medical treatment. Differences in major histocompatibility complexes (MHCs) and antigens displayed by MHCs on donor cells are the primary reasons for the acute graft rejection. Current immunosuppressive therapies, however, subdue a recipient's entire immune system, leaving the recipient at risk for pathogens, cancers, and other maladies related to the use of immunosuppressants.

There is a need in the art for new and improved allospecific immunomodulatory methods and APC-derived extracellular vesicles (APC-derived EVs) that obviate, or at least limit, exposing the patient to chronic and toxic immunosuppressive therapy in reconstructive transplantation and VCA.

### SUMMARY

Embodiments of the present disclosure generally relate to methods for fabricating extracellular vesicles. More specifically, embodiments of the disclosure relate to fabrication of extracellular vesicles derived from APCs that are engineered to dampen the immune response and prevent T cell activation against a foreign graft.

In an embodiment is provided a method for fabricating APC-derived EVs. The method includes cloning an immunomodulatory gene into a gene insertion vector, infecting antigen-presenting cells (APCs) with the gene insertion vector to form infected APCs, purifying the infected APCs, harvesting and centrifuging the infected APCs, and isolating the infected APCs.

In another embodiment is provided a method for fabricating APC-derived EVs. The method includes cloning an immunomodulatory gene into a lentiviral vector, identifying a candidate gRNA involved in a co-stimulatory pathway, a co-inhibitory receptor pathway, or a combination thereof using a gene delivery method, electroporating or infecting the candidate gRNA with a delivery system into APCs, purifying the electroporated or infected APCs (which may be genetically modified), sequencing the electroporated or infected APCs to confirm the presence of the immunomodulatory gene, a knock out of a co-stimulatory allele, or a combination thereof to identify positive cell lines, isolating APC-derived EVs with overexpressed suppressing signals, knocked out stimulating signals, or a combination thereof.

In an embodiment is provided a method for fabricating APC-derived EVs with overexpressed suppressing signals. The method includes cloning a co-inhibitory receptor into a lentiviral vector, infecting APCs with the lentiviral vector to form infected APCs, purifying the infected APCs, harvesting and centrifuging the infected APCs, and exposing the infected APCs to a reagent for isolating APC-derived EVs with overexpressed suppressing signals. Functions of the engineered APC-derived EVs in suppressing recipient lymphocyte proliferation are further tested prior to testing allospecificity in a complex mixed lymphocyte reaction (MLR) culture model.

In another embodiment, a method for fabricating APC-derived EVs with overexpressed suppressing signals is provided. The method includes cloning a co-inhibitory receptor into a lentiviral vector, infecting APCs with the vector to form infected APCs, purifying the infected APCs by fluorescent-activated cell sorting (FACS) for red fluorescent proteins (RFP), harvesting and centrifuging the infected APCs, and exposing the infected APCs to a reagent for isolating APC-derived EVs with overexpressed suppressing signals. Functions of the engineered APC-derived EVs in suppressing recipient lymphocyte proliferation are further tested prior to testing allospecificity in a complex MLR culture model.

In another embodiment is provided a method for fabricating APC-derived EVs absent stimulating signals. The method includes identifying a candidate guide ribonucleic acid (gRNA) on a co-stimulatory gene using a CRISPR design tool, electroporating the candidate gRNA into APCs to form infected APCs, purifying infected APCs for isolating co-stimulatory genes, wherein the co-stimulatory genes are removed from the infected APCs, sequencing infected APCs to confirm knock out of a co-stimulatory allele to identify positive cell lines, harvesting and centrifuging the positive cells lines, and exposing the positive cell lines to a reagent for isolating APC-derived EVs with knocked out co-stimulatory signals. The ability of the engineered APC-derived EVs to promote reduction of the proliferation of T cells in vitro are tested using a MLR culture model. Allospecificity of the APC-derived EVs are tested in a complex MLR culture model.

In another embodiment is provided a method for fabricating APC-derived EVs absent stimulating signals is provided. The method includes identifying a candidate guide gRNA on a co-stimulatory gene using a CRISPR design tool, electroporating the candidate gRNA into APCs to form infected APCs, allowing infected APCs to rest for at least 20 hours, purifying infected APCs for isolating co-stimulatory genes, wherein the co-stimulatory genes are removed from the infected APCs, sequencing infected APCs to confirm knock of a co-stimulatory allele to identify positive cell lines, harvesting and centrifuging the positive cells lines, and exposing the positive cell lines to a reagent for isolating APC-derived EVs with knocked out co-stimulatory signals. The ability of the engineered APC-derived EVs to promote reduction of the proliferation of T cells in vitro are tested using a MLR culture model. Allospecificity of the APC-derived EVs are tested in a complex MLR culture model.

In another embodiment is provided a method for fabricating APC-derived EVs with both overexpressed suppressing signals and knocked out stimulating signals. The method includes cloning a co-inhibitory receptor into a lentiviral vector, infecting APCs the lentiviral vector to form infected APCs, identifying a candidate gRNA on a co-stimulatory gene using a CRISPR design tool, electroporating the candidate gRNA into the infected APCs, purifying infected APCs for isolating co-stimulatory genes, wherein the co-stimulatory genes are removed from the infected APCs, sequencing infected APCs to confirm the presence of the co-inhibitory receptor and a knock out of a co-stimulatory allele to identify positive cell lines, harvesting and centrifuging the positive cell lines, and exposing the positive cell lines to a reagent for isolating APC-derived EVs with overexpressed suppressing signals and knocked out stimulating signals. Functionality of the engineered APC-derived EVs in suppressing recipient lymphocyte proliferation are further tested prior to testing allospecificity in a complex MLR culture model.

In at least one embodiment, a method for fabricating APC-derived EVs with both overexpressed suppressing signals and knocked out stimulating signals is provided. The method includes cloning a co-inhibitory receptor into a lentiviral vector, infecting APCs the lentiviral vector to form infected APCs, identifying a candidate gRNA on a co-stimulatory gene using a CRISPR design tool, electroporating the candidate gRNA into the infected APCs, allowing infected APCs to rest for at least 20 hours, purifying infected APCs for isolating co-stimulatory genes, wherein the co-stimulatory genes are removed from the infected APCs, sequencing infected APCs to confirm the presence of the co-inhibitory receptor and the knock out of a co-stimulatory allele to identify positive cell lines, harvesting and centrifuging the positive cell lines, and exposing the positive cell lines to a reagent for isolating APC-derived EVs with overexpressed suppressing signals and knocked out stimulating signals. Functionality of the engineered APC-derived EVs in suppressing recipient lymphocyte proliferation are further tested prior to testing allospecificity in a complex MLR culture model.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limited of its scope, may admit to other equally effective embodiments.

FIG. 1A illustrates the mechanism by which activated APC-derived EVs stimulate the activation and proliferation of T cells.

FIG. 1B is an illustration of the general concept for example engineered APC-derived EVs that display MHCs and high quantities of the co-inhibitory receptor Programmed death-1 ligand 1 (PD-L1) without cluster of differentiation 80 (CD80) according to at least one embodiment of the present disclosure.

FIG. 2A illustrates example operations for creating engineered EVs by lentiviral modification of donor-strain APCs according to at least one embodiment of the present disclosure.

FIG. 2B illustrates an example experimental design used to test whether EVs can prevent or reduce destruction of the skin tissue by allogeneic GFP+ lymphocytes according to at least one embodiment of the present disclosure.

FIG. 2C illustrates an example experimental design used to test tolerance and anergy according to at least one embodiment of the present disclosure.

FIG. 3 is an example method for fabricating engineered APC-derived EVs according to at least one embodiment of the present disclosure.

FIG. 4 is an example method for fabricating engineered APC-derived EVs according to at least one embodiment of the present disclosure.

FIG. 5 is an example method for fabricating engineered APC-derived EVs according to at least one embodiment of the present disclosure.

FIG. 6 is an example method for fabricating engineered APC-derived EVs according to at least one embodiment of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments of the present disclosure generally relate to methods for fabricating extracellular vesicles. More specifically, embodiments of the disclosure relate to fabrication of extracellular vesicles derived from APCs that are engineered to dampen the immune response and prevent T cell activation against a foreign graft. The inventors have found new and improved approaches to engineer antigen-presenting cells for the isolation of APC-derived microvesicles that are used to create antigen-specific immune tolerance in tissue transplantation. Certain embodiments provide an allospecific immunomodulatory strategy that are applied to VCA recipients and other recipients of tissue transplants. Briefly, the strategy takes advantage of the unique properties of extracellular vesicles derived from APCs.

Reconstructive transplantation and VCA restores the quality of life for individuals who have suffered devastating injuries. During transplantation, a recipient's immune system remains one of the most arduous barriers to graft transplantation. Accordingly, reconstructive transplantation and VCA are typically accompanied by an aggressive, lifelong immunosuppressive therapy that is toxic, expensive, and increases a patient's susceptibility to pathogens. Such immunosuppressive therapy includes, e.g., immunosuppressive drugs, that patients typically take to prevent rejection over the course of their lifetime. Conventional methods of immune suppression typically subdue the entire immune system, leaving the recipient at risk for pathogens, cancers, and other maladies related to use of these immunosuppressants. In contrast to such conventional methods, the new and improved allospecific immunomodulatory methods and APC-derived EVs obviate, or at least limit, exposing the patient to chronic and toxic immunosuppressive therapy in reconstructive transplantation and VCA.

Extracellular vesicles and/or extracellular-derived vesicles (collectively, EVs) are microvesicles derived from the plasma membrane of cells and are known to transfer bioactive molecules (e.g., messenger ribonucleic acid (mRNA), microRNA (miRNA), deoxyribonucleic acid (DNA), proteins, and lipids) to other cells with virus-like efficiency. As EVs are derived from the plasma membrane, they carry the same membrane-bound biomolecules (e.g., proteins, lipids, glycolipids, glycans, glycoproteins, proteoglycans, etc.) as the cell from which they are generated. The microvesicles also contain biomolecular cargos (e.g., nucleic acids, proteins, lipids, glycans, glycolipids, proteoglycans, glycoproteins, etc.). For APCs, EVs display MHC I, MHC II, and co-stimulatory receptors. Display of the MHCs and the co-stimulatory receptors on the same surface can create synergy in the signaling within T cells during T cell activation against the graft.

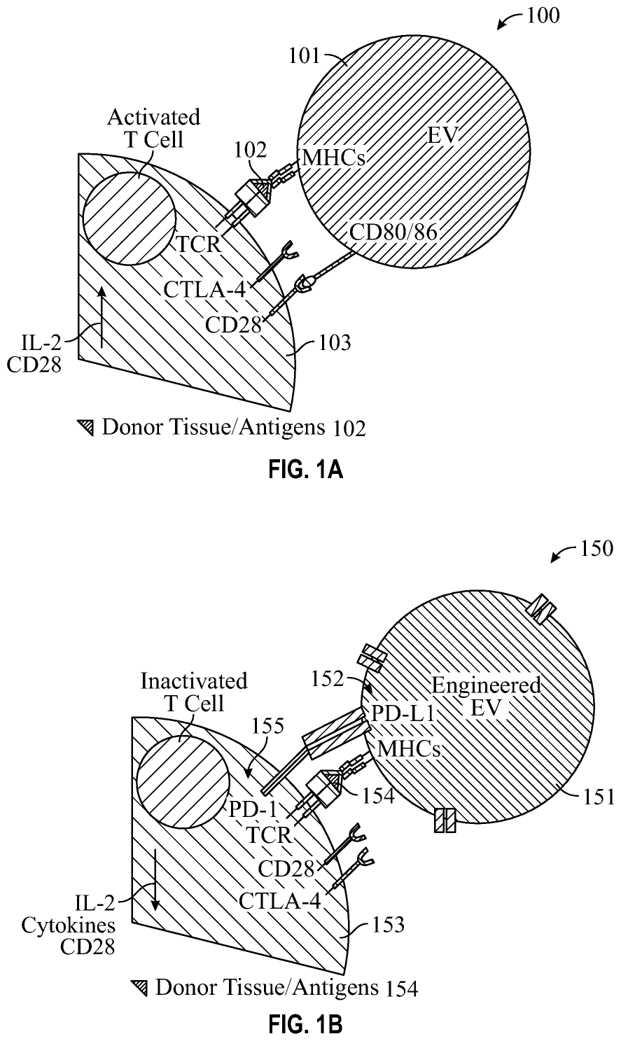

FIG. 1A illustrates a mechanism 100 by which activated APC-derived EVs stimulate the activation and proliferation of T cells. The APC-derived EVs are unmodified EVs. Here, EVs 101 from activated APCs display allogeneic MHCs together with activation-inducing co-receptors. The EVs 101 ligate to, e.g., TCR and CD28 on recipient T cells 103. In addition, the MHCs present donor antigens 102 to recipient T cells 103. The recipient T cells 103 are activated to attack the graft by APCs through direct and indirect allorecognition, utilizing MHC-TCR binding, co-stimulatory receptors (e.g., cluster of differentiation 80 (CD80)), and/or cytokines. The presence of specific co-receptors generates an allospecific activation of recipient T cells 103 against the allogeneic MHCs of the graft.

FIG. 1B is an illustration of the general concept 150 of example engineered APC-derived EVs described herein. In some examples, and as shown in FIG. 1B, the engineered APC-derived EVs display MHCs and certain quantities of the co-inhibitory receptor Programmed death-1 ligand 1 (PD-L1), and are free of CD80. A result of using the engineered EVs described herein is, at least, inactivation of the T cells.

The engineered EVs 151 ligate to various receptors on recipient T cells 153. In addition, the MHCs present donor antigens 154 to the recipient T cells 153. Briefly, the engineered EVs 151 include a PD-L1 co-inhibitory receptor 152 along with MHCs, and are free of CD80 co-stimulatory receptor. PD-L1 co-inhibitory receptor 152 is a co-inhibitory receptor present in APCs that interacts with PD-1 co-receptor 155 present in recipient T cells 153, resulting in inactivation of recipient T cells 153. CD80 (displayed by unmodified EVs 101 of FIG. 1A), in contrast, is a co-stimulatory receptor that plays a role in the initiation and maintenance of an immune response that destroys the graft. Here, then, the display of both donor MHCs and co-inhibitory receptors (e.g., PD-L1), and the absence of co-stimulatory receptors (e.g., CD80) on the same EV can prevent, or at least limit, T cell activation against the graft.

Embodiments of the present disclosure generally provide for localized or targeted immunosuppression as an alternative to chronic immunosuppression. The engineered donor APC-derived EVs display MHCs and co-inhibitory receptors on the same surface, instead of MHCs and co-stimulatory receptors on the same surface. Such engineered donor APC-derived EVs can then be infused into the recipient and modulate the recipient's immune response. The display of both donor MHCs and co-inhibitory receptors on the same EV is expected to have a synergistic effect on host T cells, preventing activation of naïve T cells against the graft and/or inducing anergy of previously activated anti-graft T cells. It is anticipated that periodic administration of the engineered EVs, a cell- and virus-free therapy, can reduce or eliminate the need for toxic immunosuppressive drugs that induce non-specific immunosuppression. Embodiments of the disclosure also include displaying PD-L1 co-inhibitory receptors along the EVs while simultaneously removing display of CD80 co-stimulatory receptors.

Embodiments described herein demonstrate an allospecific immunomodulatory strategy to avert the need for chronic, toxic immunosuppression in reconstructive transplantation and VCA. In some embodiments, a new cell- and virus-free system is created to display diverse alloantigens with tolerance-inducing stimuli. Embodiments described herein demonstrate the feasibility of a new approach used to create allospecific tolerance in tissue transplantation by engineering APCs used to fabricate microvesicles of proper biological cues. It is contemplated that the engineered APC-derived EVs have a correct set of biological cues which dampen the immune response against immune cells that are targeting a specific antigen while leaving the remainder of the immune system less affected or entirely unaffected. In at least one embodiment, APC cell lines are engineered to both overexpress co-inhibitory receptors and knock-out co-stimulatory receptors to inactivate host T cells in an allospecific manner.

Utilization of extracellular vesicles (e.g., microvesicles and/or exosomes) in an engineered delivery system to create novel immunotherapies is desirable for at least the reason that microvesicles carry the same membrane-bound biomolecules and biomolecular cargos as the cell membrane from which they are derived. The engineering method described herein, at least, alters APCs in such a way that increases or decreases the abundance of biomolecules (proteins, lipids, glycans, glycolipids, glycoproteins, proteoglycans, nucleic acids, etc.) in microvesicle membranes and/or within microvesicles that modulate the immune response. These biomolecules fall into two general categories: biomolecules that stimulate the immune system to activate and biomolecules that repress the immune system to prevent or attenuate activation. When combined with MHCs present on the surface of microvesicles, the abundance/presence of repressing or stimulating biomolecules modulates the immune system in targeted ways.

In the case of immune tolerance, the correct set of biological cues as described herein is used to dampen or tolerize the immune response of immune cells that are targeting a specific antigen expressed on specific tissues while leaving the remainder of the immune system functional. Engineered extracellular microvesicles are used to create this immune tolerance toward donor tissue. Immunotolerance is achieved by engineering donor-derived cells to produce microvesicles with increased amounts of suppressing biomolecules and/or decreasing the abundance of stimulating biomolecules. Stimulating molecules are defined as those that promote the immune system to attack and destroy cells and tissues. Suppressing biomolecules are defined as those that inhibit or lower the ability of the immune system to attack and destroy cells and tissues.

PD-L1 is an example of a suppressing biomolecule that, when increased on microvesicles that have MHCs, causes the host immune system to ignore or refrain from attacking donor-derived tissue displaying donor-derived MHCs. It is contemplated that manipulation of PD-L1 in the APC prior to harvesting can produce APC-derived EVs capable of suppressing lymphocyte proliferation in mixed lymphocyte reactions (MLRs). APC-derived EV concentrations and/or other co-inhibitory receptors (e.g., CD160, PD-L2, LAG3) with or without PD-L1 overexpression may also be modulated to influence characteristics of the engineered microvesicles.

CD80 is an example of a stimulating biomolecule. Specifically, donor-derived cells engineered to not express CD80 lack this signal. Additionally, microvesicles can have both increased amounts of suppressing biomolecules (such as PD-L1) and decreased quantities of stimulating biomolecules (such as CD80). Because APCs typically have MHC I and MHC II, APCs are particularly good cells from which engineered EVs are derived. It is contemplated that both the removal of CD80^{-/-} and the overexpression of PD-L1 in the APC prior to harvesting can produce APC-derived EVs capable of suppressing T cell proliferation in the MLR at a greater capacity than that of an EV with PD-L1 overexpressed or an EV with the removal of CD80.

### EXAMPLES

Characterization techniques of APC-derived EVs: APC-derived EVs are characterized according to criteria established by the International Society for Extracellular Vesicles for morphology (transmitted electronic microscopy), CD63 and Alix proteins (western blot), and cargo (microRNA profiling and western blot).

### Example 1

### Preparation of Unmodified APC-Derived EVs

In some embodiments, unmodified APC-derived EVs (non-engineered EVs) are fabricated to compare anergy and tolerance against engineered APC-derived EVs. Successful isolation and characterization of the unmodified EVs provides a baseline step for generating engineered APC-derived EVs. Preparation of the unmodified APC-derived EVs, in one embodiment, are performed by the following procedure. First, a non-infected APC is chosen from one of, e.g., the SSC142 APC cell line or the Raw264.7 APC cell line. Culture media from non-infected APCs is harvested and centrifuged under centrifugation conditions, e.g., a speed of rotation between about 1500×g and about 2500×g, for a time period (e.g., at least about 25 minutes) to remove cells and debris. A polymer or polymer-containing reagent, such as ExoQuick® reagent or poly(ethylene glycol) (PEG), is used to isolate APC-derived EVs.

In some embodiments, ExoQuick® reagent continues to be used while the method of ultracentrifugation is altered. It is contemplated that centrifuge methods may also be modulated to influence the quantity and quality of APC-derived EVs.

### Example 2

### Preparation of APC-Derived EVs with Overexpressed Suppressing Signals

In some embodiments, APC-derived EVs are fabricated with an increase of co-inhibitory receptor PD-L1. Preparation of the APC-derived EVs having an increase of co-inhibitory receptor PD-L1 can be performed by the following procedure. First, a non-infected APC is chosen from one of, e.g., the SSC142 APC cell line or the Raw264.7 APC cell line. To increase the expression of PD-L1 in APC-derived EVs, a lentiviral vector is prepared by cloning PD-L1 into a gene insertion vector (e.g., EF.CMV.RFP vector) and that vector is then applied to APCs for infection of the cells. Infected APCs are then purified by, e.g., fluorescent-activated cell sorting (FACS) for red fluorescent proteins (RFP). A western blot and RT-qPCR is used to verify the increased expression of PD-L1. Culture media from infected APCs is harvested and centrifuged under centrifugation conditions, e.g., a speed of rotation between about 1500×g and about 2500×g, for a time period (e.g., at least about 25 minutes) to remove cells and debris. A polymer or polymer-containing reagent, such as ExoQuick® reagent or poly(ethylene glycol) (PEG), is used to isolate APC-derived EVs for characterization. Western blotting and immunogold labeling are performed to compare the levels and localization of PD-L1 and MHCs between three experimental groups: (a) control EVs, (b) EVs from APCs infected with empty virus, and (c) EVs obtained from APCs infected with PD-L1 receptor virus.

It is contemplated that altering the promoter from CMV in the gene insertion vector (e.g., EF.CMV.RFP vector) to a promoter sequence (e.g., PKG, or another similarly strong promotor), or changing to a retrovirus may influence the quantity of co-inhibitory receptor PD-L1 on the membrane of the engineered APC-derived EV. It is further contemplated that altering the culture media conditions can promote MHC display by APCs. Media conditions are, in one embodiment, altered by the addition of, e.g., lipopolysaccharide (LPS) or phorbol 12-myristate 13-acetate (PMA). In embodiments where undesirable co-stimulatory modules are increased, removal of co-stimulatory receptors from APCs can be performed before harvesting microvesicles.

### Example 3

### Preparation of APC-Derived EVs with Reduced or Removed Stimulating Signals

In some embodiments, APC-derived EVs are fabricated to eliminate or significantly reduce the co-stimulatory receptor CD80. Preparation of the APC-derived EVs having eliminated or reduced CD80 can be performed by the following procedure.

In the procedure, CRISPR-Cas9 is used to knock out the co-receptor CD80 in unmodified APCs. The CRISPR design tool (crispr.mit.edu23) is used to identify candidate gRNA of the CD80 gene. The resulting genetic construct is electroporated into non-infected APCs. Non-infected APCs are chosen from one of, e.g., the SSC142 APC cell line or the Raw264.7 APC cell line. After, e.g., at least about 20 hours, after being electroporated, cells are sorted by, e.g., FACS to isolate Cas9+ cells based on positive fluorescence. Sequencing is performed to confirm the knockout of both alleles and identity positive cell lines. The positive cell lines are then used to obtain EVs. Culture media from positive cells lines is harvested and centrifuged under centrifugation conditions, e.g., a speed of rotation between about 1500×g and about 2500×g, for a time period (e.g., at least about 25 minutes) to remove cells and debris. A polymer or polymer-containing reagent, such as ExoQuick® reagent or poly(ethylene glycol) (PEG), is used to isolate APC-derived EVs for characterization. To confirm the knock out at the protein level, the quantity and location of CD80 from APC-derived EVs is measured by western blot and immunogold labeling.

It is contemplated that altering the culture media conditions can increase the likelihood that all or substantially all of the co-stimulatory inhibitors are removed from the engineered APC-derived EVs. Media conditions can be altered by the addition of transforming growth factor beta (TGF-β), which reduces the activation state of APCs, or the addition of microRNAs used to reduce CD80 levels in cultured APCs.

### Example 4

### Preparation of APC-Derived EVs with Overexpressed Suppressing Signals and Reduced/Removed Stimulating Signals

In some embodiments, APC-derived EVs are fabricated to overexpress co-inhibitory receptor PD-L1 and eliminate co-stimulatory receptor CD80. Preparation of the APC-derived EVs having an increased level of co-inhibitory receptor PD-L1 and a reduced level of (or eliminated) CD80 can be performed by the following procedure. First, a non-infected APC is chosen from one of, e.g., the SSC142 APC cell line or the Raw264.7 APC cell line. To increase the expression of PD-L1 in APC-derived EVs, a lentiviral vector is prepared by cloning PD-L1 into the EF.CMV.RFP vector, and that vector is then applied to APCs for infection of the cells.

Additionally, CRISPR-Cas9 is used to knock out the co-receptor CD80 in infected APCs. The CRISPR design tool (crispr.mit.edu23) is used to identify candidate gRNA of the CD80 gene. The resulting genetic construct is electroporated into infected APCs. After at least 20 hours after being electroporated, cells are purified and sorted by, e.g., FACS to isolate Cas9+ cells based on positive fluorescence. Sequencing is performed to confirm the knockout of both alleles and identify positive cell lines. The positive cell lines are then used to obtain EVs.

Culture media from positive cells lines is harvested and centrifuged under centrifugation conditions, e.g., a speed of rotation between about 1500×g and about 2500×g, for a pre-determined time period (e.g., at least about 25 minutes) to remove cells and debris. A polymer or polymer-containing reagent, such as ExoQuick® reagent or poly(ethylene glycol) (PEG), is used to isolate APC-derived EVs for characterization. Western blotting and immunogold labeling are performed to compare the levels and localization of PD-L1 and CD80.

It is contemplated that altering the promoter from CMV in the EF.CMV.RFP vector to PKG, or another similarly strong promotor, or changing to a retrovirus can influence the quantity of co-inhibitory receptor PD-L1 on the membrane of the engineered APC-derived EV. It is also contemplated that altering the culture media conditions can promote MEW display by APCs. Media conditions can be altered by the addition, e.g., of LPS or PMA. In embodiments where undesirable co-stimulatory modules are increased, removal of co-stimulatory receptors from APCs can be performed before harvesting microvesicles.

It is further contemplated that altering the culture media conditions can increase the likelihood that all or substantially all co-stimulatory inhibitors are removed from the engineered APC-derived EVs. Media conditions can be altered by the addition of, e.g., TGF-β, which reduces the activation state of APCs, or the addition of microRNAs used to reduce CD80 levels in cultured APCs.

### Example 5

### Model Testing of APC-Derived EVs

A model system is tested in mice, using C57BL/6 and BALB/c as a full MHC mismatch model. APC cell lines from donor strain C57BL/6 (SSC142 murine-APC dendritic cells) are used to generate the EVs, where BALB/c GFP+ is the recipient strain. For allospecificity experiments, FVB RFP+ mice are used, which are also a complete mismatch with C57BL/6 and BALB/c. Power analysis is conducted to determine sample size to obtain statistical significance, where data is analyzed by one-way ANOVA with a 0.05 alpha value, followed by Tukeys post-hoc test.

### Example 6

### Testing the Capability of APC-Derived EVs with Overexpressed Suppressing Signals to Suppress Recipient Lymphocyte Proliferation

Recipient lymphocytes are isolated from the spleen of donor mice (BALB/c GFP+), cultured, and used in a mixed lymphocyte reaction (MLR) culture model. The MLR is a well-used protocol that provides a simple and powerful in vitro model for the study of allogeneic T cell activation and proliferation. This protocol is adapted to test the capability of engineered EVs to suppress recipient strain T cell proliferation. The experimental groups used for these experiments are shown in Table 1. After 6 days, recipient strain GFP+ lymphocytes are collected and quantified by FACS.

TABLE 1
Groups Tested

| Experimental Group | Purpose |
|---|---|
| Donor and recipient lymphocytes | Suppression of T cell proliferation |
| with 2.5, 25, or 50 mg/mL of | by using different EVs |
| engineered EVs | concentrations with donor APCs |
| Recipient lymphocytes with 2.5, | Suppression of T cell proliferation |
| 25, or 50 mg/mL of engineered | by using different EVs |
| EVs | concentrations without donor |
| | APCs |
| Recipient lymphocytes mixed with | Baseline of T cell proliferation rate |
| stimulatory component (phorbol | (Control group) |
| 12-myristate 13-acetate (PMA) and | |
| IL-2) | |
| Recipient lymphocytes and donor | Baseline of T cell proliferation rate |
| strain mature (pre-stimulated with | when in contact with effective |
| LPS effective at presenting antigen | APCs (Control group) |
| to T cells APCs | |
| Recipient lymphocytes and donor | Baseline of T cell proliferation rate |
| strain immature APCs | when in contact with non-effective |
| | APCs (Control group) |
| Donor mature APCs and recipient | Baseline of T cell proliferation rate |
| lymphocytes with non-engineered | when in contact with non- |
| APC derived EVs at matching | engineered EVs (Control group) |
| doses | |
| Recipient lymphocytes with non- | Baseline of T cell proliferation rate |
| engineered APC-derived EVs at | when in contact with non- |
| matching doses | engineered EVs (Control group) |
| Recipient lymphocytes mixed with | Baseline of T cell proliferation rate |
| stimulatory component and with | when in contact with stimulatory |
| 2.5, 25, or 50 mg/mL of engineered | stimuli and engineered EVs |
| EVs | (Control group) |

EVs which successfully inhibit the proliferation of recipient GFP+ lymphocytes in the simple MLR are then conducted in a complex MLR to assess allospecificity. In this model, replication-competent BALB/c GFP+ recipient lymphocytes are placed in culture with replication incompetent C57BL/6 donor lymphocytes and 3rd party replication incompetent FVB RFP+ lymphocytes. Allospecific EVs can spare C57BL/6 lymphocytes while eliminating FVB RFP+ lymphocytes by host strain BALB/c GFP+ lymphocytes.

Additional metrics, such as western blot or RT-qPCR, may also be used to further classify recipient lymphocytes exposed to engineered APC-derived EVs for specific proteins and RNAs that indicate reduction in proliferation and/or anergy pathways. It is contemplated that distinguishing between donor, host, and 3rd party lymphocytes together in the same culture may become difficult when assessing allospecificity in the complex MLR. Other embodiments can use 3rd party (FVB RFP+) lymphocytes, recipient lymphocytes, and EVs derived from donor APCs which are compared against the MLRs between C57BL/6 and BALB/c GFP+.

### Example 7

### Testing the Capability of APC-Derived EVs with Removed Stimulating Signals to Suppress Recipient Lymphocyte Proliferation

To test CD80^{-/-} engineered EVs' capability in suppressing allogeneic T cell proliferation, 2.5 mg/mL, 25 mg/mL, and 50 mg/mL concentrations of engineered EVs are tested in MLR with recipient GFP+ lymphocytes. Experiment groups are similar to the experiment groups listed in Table 1 for APC-derived EVs with overexpressed suppressing signals. Engineered EVs that show suppressive activities in the simple mixed MLR are then conducted in a complex MLR to assess allospecificity. In this model, replication-competent BALB/c GFP+ recipient lymphocytes are placed in culture with replication incompetent C57BL/6 donor lymphocytes and 3rd party replication incompetent FVB RFP+ lymphocytes. Allospecific EVs can spare C57BL/6 lymphocytes while eliminating FVB RFP+ lymphocytes by host strain BALB/c GFP+ lymphocytes.

### Example 8

### Testing the Capability of Engineered APC-Derived EVs In Vitro Capacity to Induce Recipient T Cell Anergy and Tolerance

In some embodiments, the functionality of engineered APC-derived EVs is tested in vitro using an ear skin-flap and lymphocyte culture system. This system measures the level of activation and tissue rejection that occurs in the initial mixed culture model as well as determines if treatment with the engineered EVs prevents reactivation of T cells upon re-exposure to tissue from the same donor.

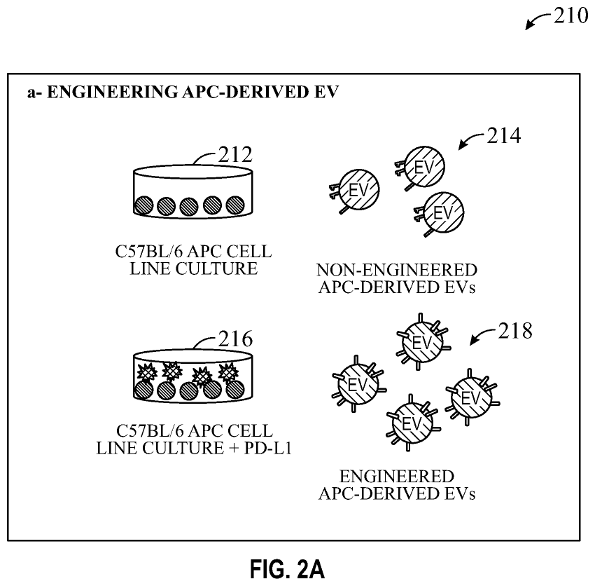

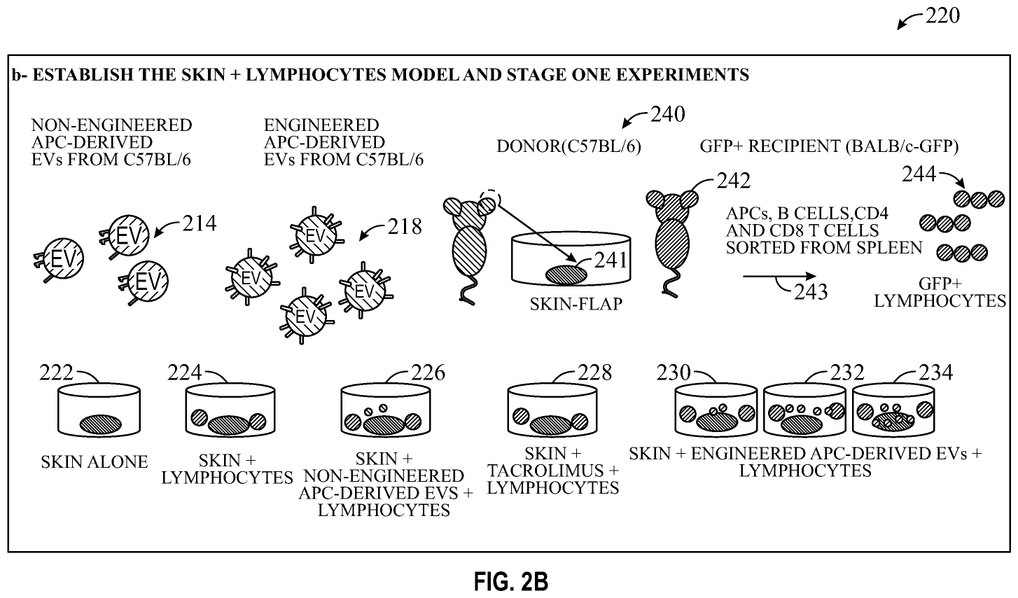

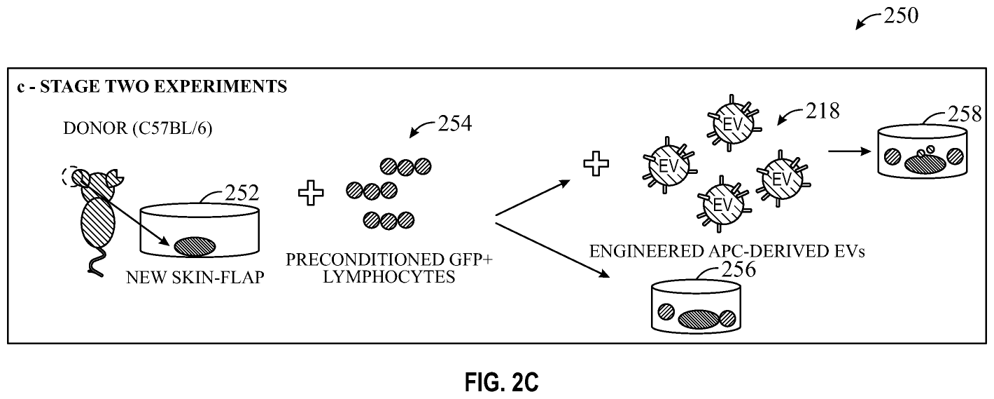

In at least one embodiment, a piece of skin from the ear from the donor (C57BL/6) is placed into culture with lymphocytes from the recipient (BALB/c GFP+), and rejection is graded, as shown in FIGS. 2A-2C. FIGS. 2A-2C collectively illustrate an overview of an example experimental design used to test engineered EVs' in vitro capacity to induce recipient T cell anergy and tolerance according to at least one embodiment of the present disclosure. The experimental design generally includes three components—(a) engineering APC-derived EV (FIG. 2A); (b) establishing the skin+lymphocytes model and stage one experiments to test, e.g., to test if EVs can prevent or reduce destruction of the skin tissue by allogeneic GFP+ lymphocytes (FIG. 2B); and (c) stage two experiments to test, e.g., tolerance and anergy (FIG. 2C).

Both the skin-flap and lymphocytes contain APCs, enabling T cell activation to occur by direct and indirect allorecognition. One advantage of using this model includes the use of a complex tissue rather than isolated cells in culture. This model additionally enables re-exposure of recipient lymphocytes to tissue from the same donor multiple times as well to tissue from a 3^{rd} party donor to test allospecificity.

To establish the model, splenic GFP+ lymphocytes 244 are isolated from recipient strain mice 242. Cells are sorted for APCs, B cells, and T cells 243 (e.g., CD4 and CD8) using the following antibodies in these combinations: CD3+CD4+, CD3+CD8+, CD19+; CD45+. Sorted lymphocytes are added into wells containing a 5 mm diameter ear skin punch obtained from adult C57BL/6 as shown in FIG. 2A. The non-engineered APC-derived EVs 214 are derived from C57BL/6 cell line culture 212, and the engineered APC-derived EVs 218 are derived from C57BL/6 cell line culture+PD-L1 216.

After about 3 and 6 days, ear skin is fixed in 10% formalin and stained with hematoxylin and eosin. Based on the severity of histopathologic changes, skin is classified for the extent of rejection from grade I to IV. Skin that has experienced severe attack by the allogeneic lymphocytes can show epidermal apoptosis, necrosis, and extensive GFP+ lymphocyte infiltration. Experimental groups to establish the model include (a) skin alone, (b) skin+lymphocytes (several replicates each with different quantities of lymphocytes), (c) skin+tacrolimus, (d) skin+tacrolimus+lymphocytes (in various amounts). The outcome of these experiments determines the most appropriate culture time and appropriate quantity of lymphocytes to achieve severe rejection.

Although conventional approaches routinely use this complex experimental setup, there is the possibility that sufficient tissue rejection will not occur. Solutions described herein which mitigate potentially undesirable results include (1) increasing the number of donor lymphocytes, (2) increasing the time in culture, (3) pre-activating recipient lymphocytes with PMA, and/or (4) pre-activating recipient strain lymphocytes with donor-strain lymphocytes from syngenetic animals or EVs isolated from mature donor APCs.

FIG. 2B illustrates an example experimental design 220 used to test whether engineered APC-derived EVs can prevent or reduce destruction of the skin tissue by allogeneic GFP+ lymphocytes according to at least one embodiment of the present disclosure. Engineered EVs are evaluated in the following experimental groups, according to FIG. 2A and FIG. 2B: (a) skin+2.5 mg/mL engineered APC-derived EVs+recipient lymphocytes 230, skin+25 mg/mL engineered APC-derived EVs+recipient lymphocytes 232, and skin+50 mg/mL of engineered APC-derived EVs+recipient lymphocytes 234. Controls include skin alone 222, skin+recipient lymphocytes 224, skin+non-engineered APC-derived EVs+recipient lymphocytes 226, skin+tacrolimus, and skin+tacrolimus+recipient lymphocytes 228. EVs with successful inhibition show histology similar to that of tacrolimus.

Furthermore, measurements of proteins from the skin by western blot related to chaperoning proteins (result of the increase of reactive oxygen species (ROS) due to injury) and proteins from GFP+ cells related to anergy and inactivation (e.g., PD-1, TCR) pathways are analyzed.

As stated above, splenic GFP+ lymphocytes 244 are isolated from recipient strain mice 242. Cells are sorted for APCs, B cells, and T cells 243 using the following antibodies in these combinations: CD3+CD4+, CD3+CD8+, CD19+; CD45+.

Where EVs show suppressive activity in these experiments, allospecificity is tested by placing ear skin from a donor (C57BL/6) and ear skin from a 3rd party (FVB RFP+) mice together in the same culture with BALB/c GFP+ lymphocytes±engineered EVs. Allospecific EVs can show rejection of FVB RFP+ skin while sparing C57BL/6 skin.

To test for allospecific T cell anergy and tolerance, stage 2 experiments 250 are conducted in accordance with FIG. 2C. In stage 2, generally, allogeneic GFP+ lymphocytes preconditioned to a specific donor skin can be re-exposed to a second piece of skin from the same donor. EVs added during re-exposure determine whether the EVs are capable of inducing allospecific tolerance.

As shown in FIG. 2C, recipient GFP+ lymphocytes 254 (which can be preconditioned) are exposed to donor ear skin 252, transferred to a new well, and re-exposed to donor skin tissue from the same donor animal. This experiment can help in determining, e.g., dose regimens. For example, whether patients can be treated from the beginning of transplant or after the transplant. Experimental groups are listed in Table 2. EVs (e.g., engineered APC-derived EVs 218) are either supplied during the primary exposure but not the secondary exposure, the second secondary but not primary exposure, or both exposures. In some examples, skin+engineered APC-derived EVs+lymphocytes 258 are the experimental group tested. Different exposures test whether the EVs provided at the first exposure induce lasting tolerance, sparing the skin during re-exposure without EVs present. Additionally, the different exposures can aid in determining whether EVs can induce anergy of T cells previously activated against the donor tissue, when EVs are supplied only upon re-exposure.

TABLE 2
Groups Tested

| Purpose | Groups Tested |
|---|---|
| Tolerance | Skin + lymphocytes + engineered APC-derived EVs applied |
| | only at stage 1 |
| Anergy | Skin + lymphocytes + engineered APC-derived EVs applied |
| | at stages 1 and 2 |
| | Skin + lymphocytes + engineered APC-derived EVs applied |
| | only at stage 2 |
| Control | Skin alone |
| | Skin + lymphocytes |
| | Skin + lymphocytes + non-engineered EVs in one or both |
| | stages |
| | Skin + lymphocytes + EVs from APCs infected with empty |
| | virus in one or both stages |
| | Skin + lymphocytes + tacrolimus control in one or both |
| | stages |

The ear skin-flap and lymphocyte culture system is first used to assess the relationship between engineered EV concentrations and tissue rejection. It is contemplated that engineered EVs reduce the tissue rejection reaction following the transplantation of tissue. Next, the ear skin-flap and lymphocyte culture system is used to test the efficacy of EVs in reducing tissue rejection upon re-exposure. Recipient GFP+ lymphocytes are exposed to donor ear skin, transferred to a new well, and re-exposed to donor skin tissue from the same donor animal. It is contemplated that the addition of EVs at first exposure can induce lasting tolerance, sparing the skin during re-exposure without EVs present. This test is also useful in determining the ability of EVs to induce anergy of T cells previously activated against the donor tissue, when EVs are supplied only upon re-exposure.

While the foregoing is directed to the addition of PD-L1 and the removal of CD80, further embodiments include (1) knocking out other co-stimulatory receptors (e.g., CD86) with or without CD80 knocked out concurrently; and/or (2) loading EVs with miRNAs known to inhibit T cell activation and proliferation. Further, while engineered APC-derived EVs suppress, induce anergy, and/or induce tolerance in recipient lymphocytes, it is contemplated that the combination of PD-L1 overexpression and CD80 knock out in EVs may, in certain instances, be insufficient to achieve anergy or tolerance; therefore, further embodiments utilize addition and/or subtraction of other receptors and/or include the use of EV cargo. Exposure to repeated dosings and/or manipulating APC-derived EV concentration is also contemplated.

Moreover, while the foregoing is directed to the fabrication of engineered microvesicles to create immune tolerance toward donor tissue, further embodiments include engineered microvesicles used to generate antigen-specific tolerance to an autoantigen mediating an autoimmune disease. Specifically, a cell (that had MHC I and/or MHC II) from the patient experiencing the autoimmune disease is engineered so that the microvesicles have MHCs displaying the autoantigen and an increased abundance of repressing biomolecules and/or decreased abundance of stimulatory molecules. Because APCs have MHC I and MHC II, APCs are particularly good cells from which engineered EVs are derived. Additionally, donor-derived cells can be engineered to increase MHC expression and/or increase the abundance of the autoantigen displayed by MHCs. It is contemplated that pulsing the cells with the autoantigen or genetically engineering the cells can produce an abundance of sufficient autoantigen displayed on MHCs.

### Example Methods

Embodiments of the disclosure generally relate to the fabrication of extracellular vesicles derived from antigen-presenting cells (APCs). In some embodiments, the APC-derived extracellular vesicles (APC-derived EVs) are engineered to dampen the immune response and prevent T cell activation against a foreign graft.

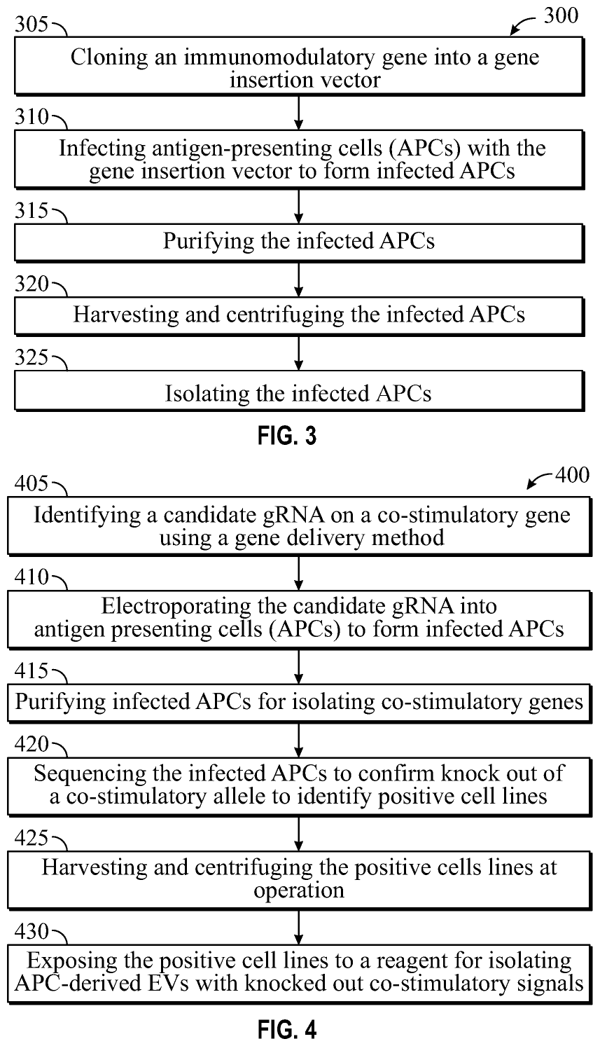

FIG. 3 is an example method 300 for fabricating engineered APC-derived extracellular vesicles (APC-derived EVs) according to at least one embodiment of the present disclosure. The method 300 includes cloning an immunomodulatory gene (e.g., a co-inhibitory receptor) into a gene insertion vector (e.g., a lentiviral vector, adenoviral vector, and/or retroviral vector) at operation 305 and infecting antigen-presenting cells (APCs) with the gene insertion vector to form infected APCs at operation 310. The infected APCs are purified at operation 315. In some embodiments, the infected APCs are purified by fluorescent-activated cell sorting (FACS) for red fluorescent proteins (RFP) or other cell sorting method. At operation 320, the infected APCs are harvested and centrifuged. The infected APCs (e.g., the APC-derived EVs) are then isolated at operation 325. Isolation can be performed by any suitable method such as exposing the infected APCs to a reagent (e.g., poly(ethylene glycol) (PEG), a derivative of PEG, or a combination thereof). The infected APC-derived EVs can have overexpressed signals.

In some embodiments, functions of the engineered EVs in suppressing recipient lymphocyte proliferation are further tested prior to testing allospecificity in a complex MLR culture model, as described above.

FIG. 4 is an example method 400 for fabricating engineered APC-derived EVs according to at least one embodiment of the present disclosure. The method 400 includes identifying a candidate gRNA on a co-stimulatory gene using a gene delivery method (e.g., a CRISPR design tool) at operation 405 and electroporating the candidate gRNA into antigen-presenting cells (APCs) to form infected APCs at operation 410. In some embodiments, the CRISPR design tool is configured to isolate Cas9+ cells. In some embodiments, the infected APCs are allowed to rest for a period of at least about 1 hour or more, such as at least about 5 hours, such as at least about 10 hours, at least about 15 hours, at least about 20 hours, at least about 25 hours, at least about 30 hours, at least about 35 hours, at least about 40 hours, at least about 45 hours, or at least about 50 hours. The infected APCs are purified for isolating co-stimulatory genes at operation 415. Here, the co-stimulatory genes are removed from the infected APCs. At operation 420, the infected APCs are sequenced to confirm knock out of a co-stimulatory allele to identify positive cell lines. The positive cell lines and harvested and centrifuged at operation 425. The positive cell lines are then exposed to a reagent for isolating APC-derived EVs with knocked out co-stimulatory signals at operation 430.

In some embodiments, the ability of the engineered EVs to promote reduction of the proliferation of T cells in vitro is tested using a MLR culture model as described above. Allospecificity of the APC-derived EVs is also tested in a complex MLR culture model.

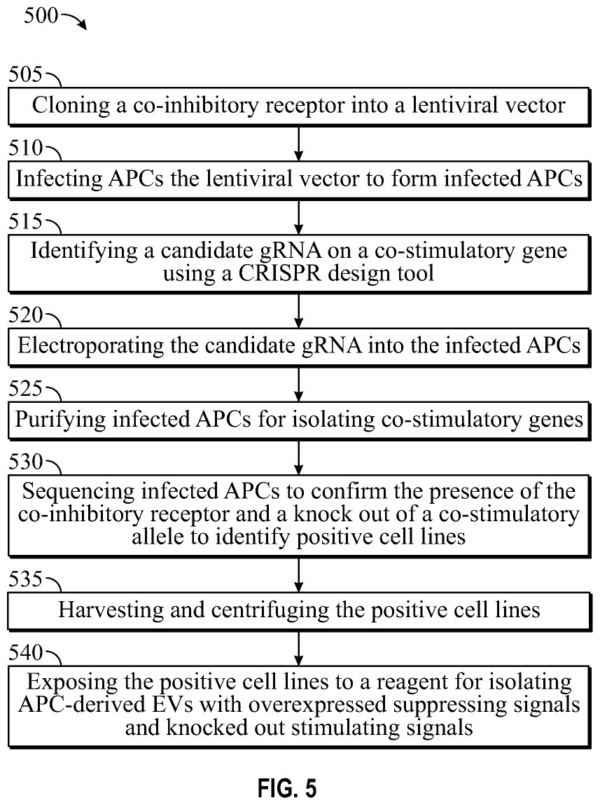

FIG. 5 is an example method 500 for fabricating engineered APC-derived EVs according to at least one embodiment of the present disclosure. The method 500 includes cloning a co-inhibitory receptor into a lentiviral vector at operation 505 and infecting antigen-presenting cells (APCs) with the lentiviral vector to form infected APCs at operation 510. The method 500 further includes identifying a candidate gRNA on a co-stimulatory gene using a CRISPR design tool at operation 515 and electroporating the candidate gRNA into the infected APCs at operation 520. In some embodiments, the infected APCs are allowed to rest for a period of at least about 1 hour or more, such as at least about 5 hours, such as at least about 10 hours, at least about 15 hours, at least about 20 hours, at least about 25 hours, at least about 30 hours, at least about 35 hours, at least about 40 hours, at least about 45 hours, or at least about 50 hours. The infected APCs are purified for isolating co-stimulatory genes at operation 525. Here, the co-stimulatory genes can be removed from the infected APCs. The infected APCs are sequenced to confirm the presence of the co-inhibitory receptor and a knock out of a co-stimulatory allele to identify positive cell lines at operation 530. At operation 535, the positive cell lines are harvested and centrifuged. The positive cell lines are then exposed to a reagent for isolating APC-derived EVs with overexpressed suppressing signals and knocked out stimulating signals at operation 540.

In some embodiments, the functionality of the engineered EVs in suppressing recipient lymphocyte proliferation is further tested prior to testing the allospecificity in a complex MLR culture model, as described above.

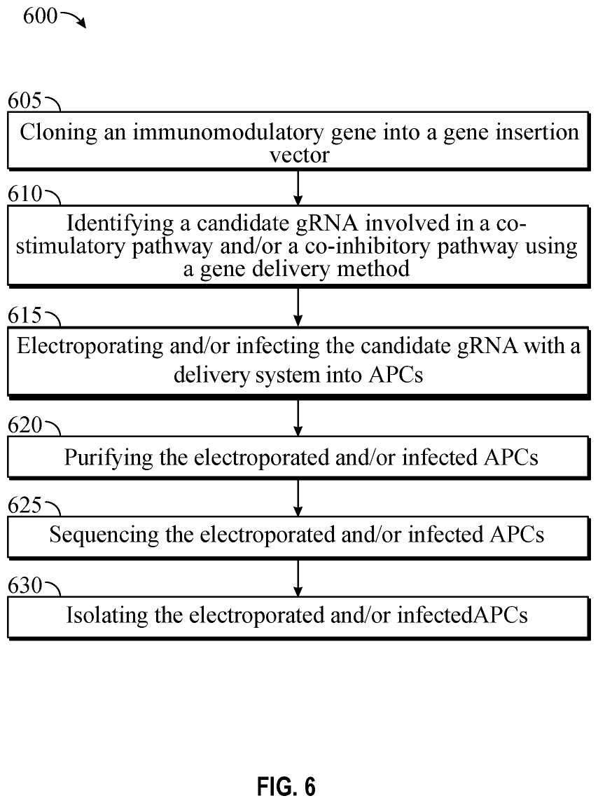

FIG. 6 is an example method 600 for fabricating engineered APC-derived EVs according to at least one embodiment of the present disclosure. Method 600 includes cloning an immunomodulatory gene into a gene insertion vector (e.g., a lentiviral vector, adenoviral vector, and/or retroviral vector) at operation 605, and identifying a candidate gRNA involved in a co-stimulatory pathway, a co-inhibitory receptor pathway, or a combination thereof using a gene delivery method (e.g., a CRISPR design tool) at operation 610. Method 600 further includes electroporating and/or infecting the candidate gRNA with a delivery system into APCs at operation 615, and purifying the electroporated and/or infected APCs (which may be genetically modified) at operation 620. The electroporated and/or infected APCs are then sequenced to confirm the presence of the immunomodulatory gene, a knock out of a co-stimulatory allele, or a combination thereof to identify positive cell lines at operation 625, and the electroporated and/or infected APCs (e.g., the APC-derived EVs) are isolated at operation 630. The APC-derived EVs can have overexpressed suppressing signals and/or knocked out stimulating signals.

In some embodiments, a method to create immunological tolerance to a donor tissue includes engineering donor-origin antigen presenting cells (APCs) by introducing genetic material that encode immunomodulatory factors into the APCs, and/or engineering donor-origin APCs by inhibiting effective production of factors that would promote host immune cells to target and destroy donor tissue. The method further includes purifying the donor-origin APCs with the immunomodulatory genes and/or inhibitory factors, and harvesting and purifying extracellular vesicles derived from the APCs. The method further includes administering the EVs to recipients of the donor's tissue to create immunological tolerance to the donor tissue.

In at least one embodiment described herein, immunomodulatory genes include, but are not limited to, receptors (e.g., PD-L1, CD160, PD-L2, LAG3, CTLA4), cytokines (e.g., IL-10, TGF-β), miRNAs (e.g., miR31, miR223, miR146a), intracellular signaling molecules (e.g., BLIMP1, mTOR) or other immunomodulatory genes singly, or a combination thereof. In some embodiments, factors that promote the host immune cells to target and destroy the donor tissue include, but are not limited to, co-stimulatory receptors (e.g., CD80/86, CD40/CD40L), cytokines (e.g., IL-12, IL-6, IL-1, IFNγ, TNFα, IL-2), miRNAs (e.g., miR150, miR155, miR214), intracellular signaling molecules (e.g., Tbet, Eomes, PTEN), or other immunomodulatory factors singly, or a combination thereof.

In some embodiments described herein, methods of gene introduction include, but are not limited to, lentiviral vector (e.g., a EF.CMV.RFP lentiviral vector or an EF.PGK.RFP lentiviral vector), adenoviral vector, retroviral vector, electroporation of genetic material, which may contain a fluorescent marker. Further, and in at least one embodiment described herein, APCs can be of donor origin, or of origin genetically identical or similar (e.g., familial) to the donor. For example, the APCs can be SSC142 APC cell line or a Raw264.7 APC cell line.

In some embodiments described herein, APCs engineered by the introduction of immunomodulatory genes are purified by fluorescent-activated cell sorting (FACS) for the fluorescent marker (e.g., red fluorescent proteins (RFP) or other cell sorting method. In at least one embodiment described herein, EVs from engineered APCs are isolated by one or more isolation methods. Isolation methods include, but are not limited to, centrifugation and/or use of a reagent (e.g., poly(ethylene glycol) (PEG) and/or a derivative of PEG, such as Exoquick® reagent).

As described herein, methods for fabricating extracellular vesicles derived from antigen-presenting cells (APCs) are used to dampen the immune response and prevent T cell activation against a foreign graft. The APC-derived EVs can create antigen-specific immune tolerance in tissue transplantation. In contrast to such conventional methods, the new and improved allospecific immunomodulatory methods and APC-derived EVs obviate, or at least limit, exposing the patient to chronic and toxic immunosuppressive therapy in reconstructive transplantation and VCA.

In the foregoing, reference is made to embodiments of the disclosure. However, it should be understood that the disclosure is not limited to specific described embodiments. Instead, any combination of the following features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the foregoing aspects, features, embodiments and advantages are merely illustrative and are not considered elements or limitations of the appended claims except where explicitly recited in a claim(s). Likewise, reference to “the disclosure” shall not be construed as a generalization of any inventive subject matter disclosed herein and shall not be considered to be an element or limitation of the appended claims except where explicitly recited in a claim(s).

For purposes of this present disclosure, and unless otherwise specified, all numerical values within the detailed description and the claims herein are modified by “about” or “approximately” the indicated value, and consider experimental error and variations that would be expected by a person having ordinary skill in the art. For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

As used herein, the indefinite article “a” or “an” shall mean “at least one” unless specified to the contrary or the context clearly indicates otherwise.

While the foregoing is directed to embodiments of the present disclosure, other and further embodiments of the disclosure may be devised without departing from the basic scope thereof, and the scope thereof is determined by the claims that follow.

What is claimed is:

1. A method for fabricating engineered antigen-presenting cell-derived extracellular vesicles (engineered APC-derived EVs), comprising:
cloning an immunomodulatory gene into a gene insertion vector;
infecting antigen-presenting cells (APCs) with the gene insertion vector to form infected APCs;
purifying the infected APCs;
harvesting and centrifuging the infected APCs; and
isolating the infected APCs.

2. The method of claim 1, wherein the immunomodulatory gene comprises one or more of receptors, cytokines, miRNAs, intracellular signaling molecules, or a combination thereof.

3. The method of claim 1, wherein the gene insertion vector comprises a lentiviral vector, an adenoviral vector, a retroviral vector, or a combination thereof.

4. The method of claim 1, wherein the APCs are of donor origin or of an origin genetically identical or similar to a donor.

5. The method of claim 1, wherein the infected APCs are purified by fluorescent-activated cell sorting (FACS).

6. The method of claim 1, wherein the infected APCs are centrifuged at a speed of rotation between about 1500×g and about 2500×g.

7. The method of claim 1, wherein the infected APCs are isolated by a reagent, the reagent comprising poly(ethylene glycol) (PEG), a derivative of PEG, or a combination thereof.

8. A method for fabricating engineered antigen-presenting cell-derived vesicles (engineered APC-derived EVs), comprising:
identifying a candidate gRNA on a co-stimulatory gene using a gene delivery method;
electroporating the candidate gRNA into antigen-presenting cells (APCs) to form infected APCs;
purifying infected APCs for isolating co-stimulatory genes, wherein the co-stimulatory genes are removed from the infected APCs;
sequencing infected APCs to confirm knock out of a co-stimulatory allele to identify positive cell lines;
harvesting and centrifuging the positive cells lines; and
exposing the positive cell lines to a reagent for isolating APC-derived EVs with knocked out co-stimulatory signals.

9. The method of claim 8, wherein the co-stimulatory gene is CD80, CD86, CD40, or combinations thereof.

10. The method of claim 8, wherein the gene delivery method is a CRISPR design tool configured to isolate Cas9+ cells.

11. The method of claim 8, wherein the APCs are of donor origin or of an origin genetically identical or similar to a donor.

12. The method of claim 8, wherein infected APCs are purified by fluorescent-activated cell sorting (FACS) configured to isolate co-stimulatory cells based on positive fluorescence.

13. The method of claim 8, wherein the infected APCs are centrifuged at a speed of rotation between about 1500×g and about 2500×g.

14. The method of claim 8, wherein the infected APCs are isolated by using a reagent, the reagent comprising poly(ethylene glycol) (PEG), a derivative of PEG, or a combination thereof.

15. A method for fabricating engineered antigen-presenting cell-derived vesicles (engineered APC-derived EVs), comprising:
cloning an immunomodulatory gene into a lentiviral vector;
identifying a candidate gRNA involved in a co-stimulatory pathway, a co-inhibitory receptor pathway, or a combination thereof using a gene delivery method;
electroporating or infecting the candidate gRNA with a delivery system into APCs;
purifying the electroporated or infected APCs;
sequencing the electroporated or infected APCs to confirm the presence of the immunomodulatory gene, a knock out of a co-stimulatory allele, or a combination thereof to identify positive cell lines; and
isolating APC-derived EVs with overexpressed suppressing signals, knocked out stimulating signals, or a combination thereof.

16. The method of claim 15, wherein the immunomodulatory gene comprises one or more of receptors, cytokines, miRNAs, intracellular signaling molecules, or a combination thereof.

17. The method of claim 15, wherein the co-stimulatory pathway involves CD80, CD86, CD40, or combinations thereof, and the inhibitory receptor pathway involves PD-L1, CD160, PD-L2, LAG3, CTLA4, or combinations thereof.

18. The method of claim 15, wherein the antigen-presenting cells are of donor origin or of an origin genetically identical or similar to a donor.

19. The method of claim 15, wherein the APCs are centrifuged at a speed of rotation between about 1500×g and about 2500×g.

20. The method of claim 15, wherein the infected APCs are isolated by using a reagent, the reagent comprising poly(ethylene glycol) (PEG), a derivative of PEG, or a combination thereof.
